(19) United States
(12) Patent Application Publication
(10) Pub. No.: US 2012/0183631 A1
(43) Pub. Date: Jul. 19, 2012

(54) INGREDIENT EXTRACTED FROM POUZOLZIA ZEYLANICA (L.) BENN AND ITS ANTI-ACNE FORMULA

(76) Inventors: A-Shiou Hung-Chen, Pingtung County (TW); Shang-Chia Liu, Taipei City (TW)

(21) Appl. No.: 13/006,445

(22) Filed: Jan. 14, 2011

(51) Int. Cl. A61K 36/185; A61P 17/10

(52) U.S. Cl. 424/725

(57) ABSTRACT

An ingredient extracted from Pouzolzia zeylanica (L.) Benn (0.00002%˜1% (w/w)) and its anti-acne formula for a purpose of anti-acne; the method to manufacture the ingredient is Pouzolzia zeylanica (L.) Benn with deionized or distilled water added is ground to develop a Pouzolzia zeylanica (L.) Benn solution and further filtered to become a Pouzolzia zeylanica (L.) Benn extract which is deposited in a low-temperature and dry environment (temperature between −20° C. and −48° C.; pressure between 10 K Bar and 133 K Bar) for development of an extract with a powdered anti-acne ingredient; additionally, the ingredient with citronella (0.5%˜1.5% (w/w)), compound disinfectant (0.3%˜1% (w/w)), and surfactant (0.5%˜3% (w/w)) added can become a pasty anti-acne formula for long-term storage.

### BACKGROUND OF THE INVENTION

1) Field of the Invention

The present invention relates to a natural anti-acne ingredient extracted from a plant, especially to an ingredient extracted from Pouzolzia zeylanica (L.) Benn, and its anti-acne formula.

2) Description of the Prior Art

Acnes attribute to metabolism disturbance of sebum which should be periodically secreted by sebaceous glands and lifted on skin via pilosebaceous ducts as one layer of natural protection membrane; however, acnes will further deteriorate when dead skin cells owing to sebum excessively secreted by sebaceous glands (probably another indirect factor such as disturbance of hormone secretion) stimulating hyperkeratinization of pilosebaceous ducts are surrounded by too much sebum and adhere each other to develop cuticula blockading skin pores; other reasons resulting in acnes generated might be an individual's diathesis, face incompletely cleaned, inadequate cosmetics, or skin infected by Propionibacterium acnes; acnes usually distribute on some specific areas such as face, breast, or upper back with sebaceous glands densely concentrating because hair pores exceptionally keratinized or blockaded with skin not properly cleaned are easily developed to acnes or dermatitis.

The general medical anti-acne articles are divided into anti-acne foaming cleanser, exfoliation product, pore cleanser, anti-acne patch and acne drug, which deliver anti-acne effect by some chemical ingredients in media touching skin except a pore cleanser relying on a physical method, and comprise probable chemical ingredients shown as follows:

### (1) Salicylic Acid

Salicylic acid with an alias of B-Hydroxy Acid (BHA) is one decorticating agent, which is effective in skin decorticated similar to effect of fruit acid but capable of permeating more cell walls than fruit acid, and delivers a purulent wound (especially a serious acne pustulosa) rapidly callused, dried and shed;

### (2) Sulfur

Sulfur is a sulfurous ingredient, which is effective in drying and decorticating acne pustulosa, and causes phenomenon similar to that with salicylic acid applied such as skin too dry or uncomfortable and skin deteriorated;

### (3) Benzoyl Peroxide

With dual effects like sterilization and cuticula dissolved, benzoyl peroxide is effective in curing both papules and inflamed acnes or moderates developed acnes within three weeks or so owing to its function to dissolve cuticula, but makes skin slightly dry and decorticated or even some people hypersensitive with symptoms like repeated irritation, extended inflammation and vesicle;

### (4) Triclosan

Triclosan, a chemical substance with the chloric structure and also an ingredient for deodorization and sterilization, is applicable to germicides mostly and other purposes extensively such as medicated soap, shower gel, and dishwashing liquid; triclosan (0.1% (w/w)) is effective in treating bacterial acnes only rather than inflamed or purulent acnes and makes an affected part dry or shed without acnes continuously deteriorating despite its sterilization to eliminate Propionibacterium acnes parasitizing in hair pores on a face;
   (5) Vitamin A Acid (Retinnoic acid or Retin-A)
Vitamin A acid featuring its excellent capability to permeate skin is capable of entering hair follicle walls to dissolve keratinized cells or infiltrates hair pores to dissolve and void cuticula with black comedo significantly decreased by three to five weeks and further totally eliminated by three months, but is inapplicable to flamed or purulent skin owing to irritation to skin.

Despite symptoms of acnes cured with the said chemical ingredients, skin (especially inflamed or purulent skin with wounds around affected parts) on which acnes are developed is always sensitive and further deteriorates due to improper treatment or contact with dirty air (or unclean objects), or is hurt by the said chemical anti-acne substances to develop dryness, strain, or cuticula shed but significantly recovered with the anti-acne substances applied for a long period and metabolism of skin activated.

### SUMMARY OF THE INVENTION

The first object of the invention: Both an ingredient extracted from Pouzolzia zeylanica (L.) Benn and its anti-acne formula without any side effect do not irritate skin wherein the anti-acne formula originates from an ingredient, which is extracted from Pouzolzia zeylanica (L.) Benn, with citronella (0.5%˜1.5% (w/w)), compound disinfectant (0.3%˜1% (w/w)) and surfactant (0.5%˜3% (w/w)) added for development of a fragrant bactericidal ointment which can be stored for a long period; the present invention as a significant breakthrough does not irritate skin in consideration of the fact that skin (especially inflamed or purulent skin with wounds around affected parts) on which acnes are developed is sensitive or further deteriorates and develop dryness, strain, or cuticula shed due to application of other chemically irritative anti-acne substances.

The second object of the invention: An ingredient extracted from Pouzolzia zeylanica (L.) Benn (0.00002%˜1% (w/w)) and its anti-acne formula with fast effect are manufactured with the following steps:

(1) Development of Pouzolzia zeylanica (L.) Benn extract: Leaves of Pouzolzia zeylanica (L.) Benn with deionized or distilled water added are ground to develop a Pouzolzia zeylanica (L.) Benn solution which is further filtered to get a Pouzolzia zeylanica (L.) Benn extract;
(2) Manufacture of an anti-acne formula: The Pouzolzia zeylanica (L.) Benn extract is deposited in a low-temperature and dry environment (temperature between −20° C. and −48° C.; pressure between 10 K Bar and 133 K Bar) for development of a powdered anti-acne formula which is favorable to storage and portability;
(3) Development of an anti-acne solution: The powdered anti-acne formula with deionized or distilled water added is fully agitated and mixed to develop an anti-acne solution for direct application to acnes on skin and becomes effective in one or two weeks only that is characteristic of neither a restriction of general pharmaceuticals continuously applied for three or five weeks prior to a symptom cured (acnes effectively treated in the beginning) nor any scar left on skin.

### DETAILED DESCRIPTIONS OF THE PREFERRED EMBODIMENTS

Pouzolzia zeylanica (L.) Benn (scientific name) with aliases of Ceylon Pouzolzia or Mist Vine is mostly produced on warm humid areas at low (medium)-altitude open fields or beside farmlands and ditches in several provinces south of the Yangtze River, China, and Taiwan, and has its leaves pounded to pieces for application to either skin with swollen poison or teeth; however, acnes are not common swellings but wounds on affected parts of sensitive skin because dead skin cells owing to sebum excessively secreted by sebaceous glands stimulating hyperkeratinization of pilosebaceous ducts are surrounded by too much sebum and adhere each other to develop cuticula blockading skin pores; the present invention is based on a Pouzolzia zeylanica (L.) Benn extract to deliver neither side effect nor skin irritated for a purpose effective in one or two weeks; first referring to FIG. 1 which indicates a flow diagram for the present invention clearly interprets an ingredient extracted from Pouzolzia zeylanica (L.) Benn (0.00002%˜1% (w/w)) and its anti-acne formula with anti-acne effect and manufacturing steps as follows:
(1) Development of a Pouzolzia zeylanica (L.) Benn extract: Leaves of Pouzolzia zeylanica (L.) Benn with deionized or distilled water added are ground to develop a Pouzolzia zeylanica (L.) Benn solution which is further filtered to get a Pouzolzia zeylanica (L.) Benn extract;
(2) Manufacture of an anti-acne formula: The Pouzolzia zeylanica (L.) Benn extract is deposited in a low-temperature and dry environment (temperature between −20° C. and −48° C.; pressure between 10 K Bar and 133 K Bar) for development of a powdered anti-acne formula which is mixed with citronella (0.5%˜1.5% (w/w)), compound disinfectant (0.3%˜1% (w/w)), and surfactant (0.5%˜3% (w/w)) for development of a fragrant bactericidal ointment which can be stored for a long period;
(3) Development of an anti-acne solution: The powdered anti-acne formula with deionized (or distilled) water or other solvents added is fully agitated and mixed to develop an anti-acne solution which is directly applied to acnes on skin

For the purpose of the anti-acne formula of the present invention conveniently used and stored, citronella (0.5%˜1.5% (w/w)), compound disinfectant (0.3%˜1% (w/w)), and surfactant (0.5%˜3% (w/w)) can be added into the anti-acne ingredient for development of a fragrant bactericidal ointment featuring long-term storage, an anti-acne foaming cleanser with other media added, an adhesive/removable face membrane (sheet, gel, lactescence or paste), or an artificial skin dressing such as anti-acne patch by which an ingredient extracted from Pouzolzia zeylanica (L.) Benn and its anti-acne formula in the present invention can be applied to different articles in daily use for extensive application and developed acnes effectively treated in the beginning without scars left on skin.

As one root cause to generate acnes, endocrine dyscrasia induces endotoxin accumulated but not regularly metabolized due to dry/hot diet, constipation, work & rest irregular, and pressure. In contrast to defecation for metabolism with food digested, feces not regularly defecated via large intestine and anus will cause toxin to be spread around the body and finally diffused to the face. In consideration of daily pressures aggravating endotoxin not effectively moderated in the body, the inventor performed experiments with two units of cells for the present invention of an ingredient extracted from Pouzolzia zeylanica (L.) Benn and its anti-acne formula wherein four samples of endotoxin (10 μg/ml) with four corresponding Pouzolzia zeylanica (L.) Benn solutions (100, 50, 25, 12.5 and 6.25 μg/m) added were compared to samples of endotoxin with different contents of Curcumin mixed and samples of Dimethyl sulfoxide (control group). As shown in experimental data of Tables 1 & 2& 3& 4, it can be seen endotoxin not restrained by Curcumin is proportionally inhibited with the contents of Pouzolzia zeylanica (L.) Benn in solutions increased (toxin inhibiting rate gradually decreased), and is still inhibited in the solution with the concentration of Pouzolzia zeylanica (L.) Benn at 6.25 μg/ml. The effects of the present invention of an ingredient extracted from Pouzolzia zeylanica (L.) Benn and its anti-acne formula are expressed in statistic results such as the distributed data with the regression analysis made (FIG. 2) and the curves of toxin inhibiting rates (FIG. 3).

TABLE 1

| | (Control | | | |
|---|---|---|---|---|
| | Group) | | Endotoxin + | Endotoxin + |
| | Dimethyl | Endotoxin | Curcumin | Curcumin |
| Sample | sulfoxide | 10 μg/ml | (1 μg/ml) | (10 μg/ml) |
| 1 | 0.1535 | 0.2755 | 0.2766 | 0.1888 |
| 2 | 0.1513 | 0.284 | 0.2707 | 0.3321 |
| 3 | 0.2753 | 0.2649 | 0.2642 | 0.1921 |
| 4 | 0.1517 | 0.2831 | 0.2793 | 0.2143 |
| Average | 0.183 | 0.2809 | 0.2755 | 0.2462 |
| Standard | 0.0616 | 0.0088 | 0.0081 | 0.0139 |
| Deviation | | | | |
| Regression | −47.63 | 17.64 | 14.09 | −5.49 |
| Line | | | | |
| Toxin Inhibiting | | | | |
| Rate | | | | |

TABLE 2

| | Pouzolzia | Pouzolzia | Pouzolzia | Pouzolzia | Pouzolzia |
|---|---|---|---|---|---|
| | zeylanica | zeylanica | zeylanica | zeylanica | zeylanica |
| | (L.) Benn | (L.) Benn | (L.) Benn | (L.) Benn | (L.) Benn |
| | solution | solution | solution | solution | solution |
| Sample | (100 μg/mL) | (50 μg/mL) | (25 μg/mL) | (12.5 μg/mL) | (6.25 μg/mL) |
| 1 | 0.2913 | 0.2879 | 0.2739 | 0.2515 | 0.2456 |
| 2 | 0.2858 | 0.2697 | 0.2695 | 0.2736 | 0.2605 |
| 3 | 0.2833 | 0.3061 | 0.2961 | 0.2684 | 0.251 |
| 4 | 0.2944 | 0.2689 | 0.2592 | 0.2563 | 0.2454 |
| Average | 0.2878 | 0.2832 | 0.2747 | 0.2661 | 0.2524 |
| Standard | 0.0057 | 0.0176 | 0.0156 | 0.0103 | 0.0032 |
| Deviation | | | | | |
| Regression | 22.29 | 19.17 | 13.52 | 7.8 | −1.36 |
| Line | | | | | |
| Toxin | −26.32 | −8.63 | 23.39 | 55.79 | 107.68 |
| Inhibiting | | | | | |
| Rate | | | | | |

TABLE 3
Pouzolzia zeylanica (L.) Benn (2)

| | (Control | | | |
|---|---|---|---|---|
| | Group) | | Endotoxin + | Endotoxin + |
| | Dimethyl | Endotoxin | Curcumin | Curcumin |
| Sample | sulfoxide | 10 μg/ml | (1 μg/ml) | (10 μg/ml) |
| 1 | 0.1535 | 0.2755 | 0.2766 | 0.1888 |
| 2 | 0.1513 | 0.284 | 0.2707 | 0.3321 |
| 3 | 0.2753 | 0.2649 | 0.2642 | 0.1921 |
| 4 | 0.1517 | 0.2831 | 0.2793 | 0.2143 |
| Average | 0.183 | 0.2809 | 0.2755 | 0.2462 |
| Standard | 0.0616 | 0.0088 | 0.0081 | 0.0139 |
| Deviation | | | | |
| Regression | −47.63 | 17.64 | 14.09 | −5.49 |
| Line | | | | |
| Toxin Inhibiting | | | | |
| Rate | | | | |

TABLE 4

| | Pouzolzia | Pouzolzia | Pouzolzia | Pouzolzia | Pouzolzia |
|---|---|---|---|---|---|
| | zeylanica | zeylanica | zeylanica | zeylanica | zeylanica |
| | (L.) Benn | (L.) Benn | (L.) Benn | (L.) Benn | (L.) Benn |
| | solution | solution | solution | solution | solution |
| Sample | (100 μg/mL) | (50 μg/mL) | (25 μg/mL) | (12.5 μg/mL) | (6.25 μg/mL) |
| 1 | 0.2719 | 0.2722 | 0.2624 | 0.2708 | 0.2593 |
| 2 | 0.2802 | 0.2654 | 0.2712 | 0.2646 | 0.2849 |
| 3 | 0.29010001 | 0.2692 | 0.2782 | 0.2748 | 0.2643 |
| 4 | 0.2771 | 0.2755 | 0.2703 | 0.2662 | 0.2647 |
| Average | 0.2798 | 0.2723 | 0.2705 | 0.2691 | 0.2628 |
| Standard | 0.0068 | 0.0051 | 0.0043 | 0.0055 | 0.0118 |
| Deviation | | | | | |
| Regression | 16.95 | 11.93 | 10.75 | 9.8 | 5.58 |
| Line | | | | | |
| Toxin | 3.94 | 32.37 | 39.07 | 44.46 | 68.39 |
| Inhibiting | | | | | |
| Rate | | | | | |

The present invention of an ingredient extracted from Pouzolzia zeylanica (L.) Benn and its anti-acne formula is effective in fast anti-acne within one or two weeks without a restriction in time like general pharmaceuticals constantly applied to acnes for three or five weeks before healing and has good effect on early acnes without scars left on skin; the inventor performed experiments to demonstrate the capability to fast remove acnes by the ingredient extracted from Pouzolzia zeylanica (L.) Benn and its anti-acne formula from March to October 2010 (Table 5): (1) Mild symptom: Acnes removed in 4 days or so; (2) Moderate symptom: Acnes removed in 8 days or so; (3) Severe symptom: Acnes removed in 15 days or so.

TABLE 5

| | | | | Extract | | Days with |
|---|---|---|---|---|---|---|
| | Test | Symp- | Quantity & | Concentra- | | Acnes |
| No. | Date | tom | Status | tion | Effect | Eliminated |
| 1 | 1999 Mar. 5 | Mild | Less than 5; | 1-1000000 | Acnes totally | 5 |
| | | | early acne | ppm | removed | |
| 2 | 1999 Jun. 5 | Mild | Less than 5; | 1-1000000 | Acnes totally | 3 |
| | | | early acne | ppm | removed | |
| 3 | 1999 Aug. 18 | Mild | Less than 5; | 1-1000000 | Acnes totally | 3 |
| | | | early acne | ppm | removed | |
| 4 | 1999 Sep. 20 | Mild | Less than 5; | 1-1000000 | Acnes totally | 5 |
| | | | early acne | ppm | removed | |
| 5 | 1999 May 8 | Mod- | Between 6 and | 1-1000000 | Acnes removed | 8 |
| | | erate | 15; acne | ppm | with the formula | |
| | | | pustulosa | | applied at two | |
| | | | (minority) | | phases | |
| 6 | 1999 Jul. 20 | Mod- | Between 6 and | 1-1000000 | Acnes removed | 8 |
| | | erate | 15; acne | ppm | with the formula | |
| | | | pustulosa | | applied at two | |
| | | | (minority) | | phases | |
| 7 | 1999 Aug. 19 | Mod- | Between 6 and | 1-1000000 | Acnes removed | 9 |
| | | erate | 15; acne | ppm | with the formula | |
| | | | pustulosa | | applied at two | |
| | | | (minority) | | phases | |
| 8 | 1999 Sep. 15 | Mod- | Between 6 and | 1-1000000 | Acnes removed | 9 |
| | | erate | 15; acne | ppm | with the formula | |
| | | | pustulosa | | applied at two | |
| | | | (minority) | | phases | |
| 9 | 1999 Jul. 22 | Severe | More than 15; | 1-1000000 | Acnes removed | 16 |
| | | | acne | ppm | with the formula | |
| | | | pustulosa | | applied at two | |
| | | | (majority) | | phases | |
| 10 | 1999 Aug. 10 | Severe | More than 15; | 1-1000000 | Acnes removed | 14 |
| | | | acne | ppm | with the formula | |
| | | | pustulosa | | applied at two | |
| | | | (majority) | | phases | |
| 11 | 1999 Sep. 8 | Severe | More than 15; | 1-1000000 | Acnes removed | 15 |
| | | | acne | ppm | with the formula | |
| | | | pustulosa | | applied at two | |
| | | | (majority) | | phases | |

### BRIEF DESCRIPTION OF THE DRAWINGS

FIG. 1 indicates the flow diagram of the present invention.

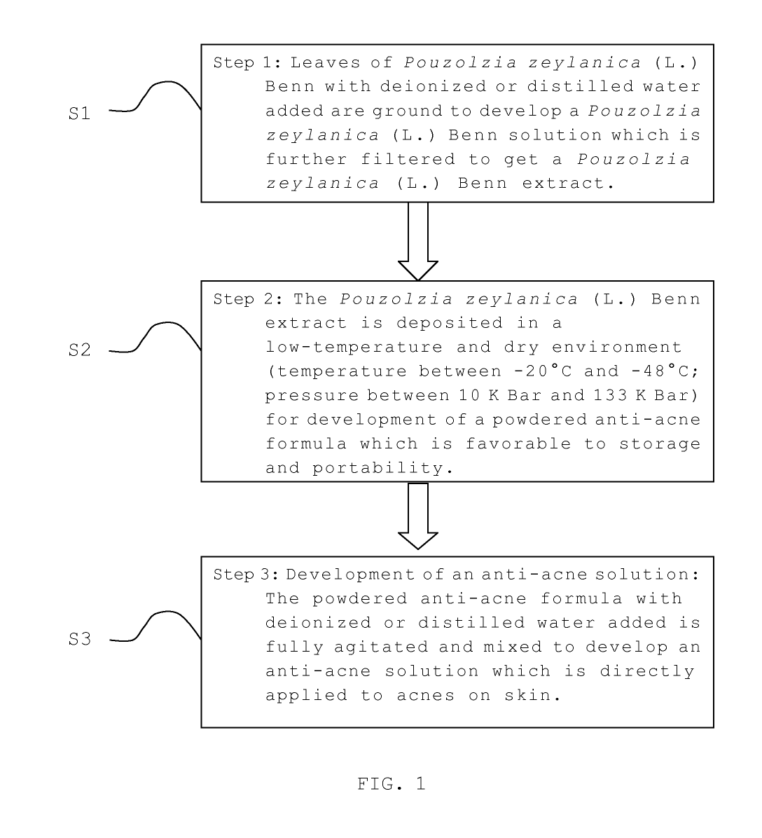

FIG. 2 indicates the distributed data with the regression analysis made for the present invention.

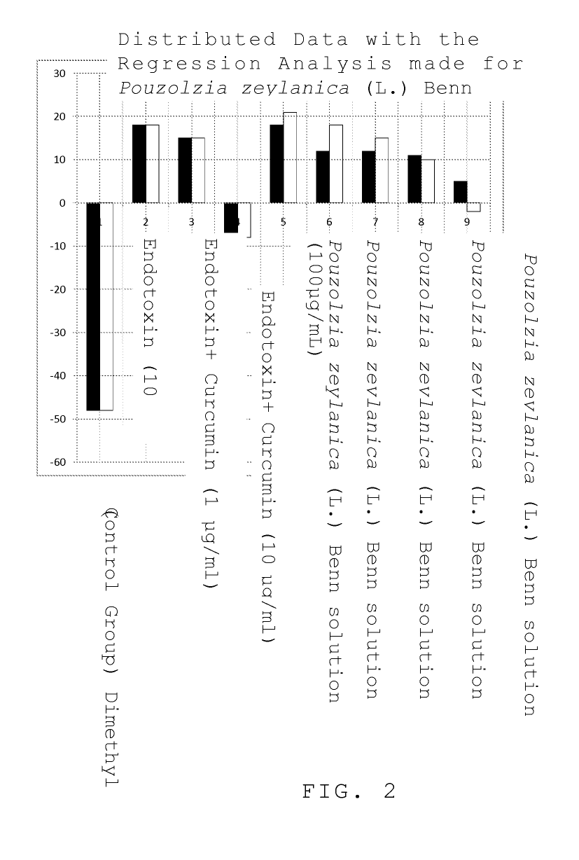

FIG. 3 indicates the curves of toxin inhibiting rates for the present invention.

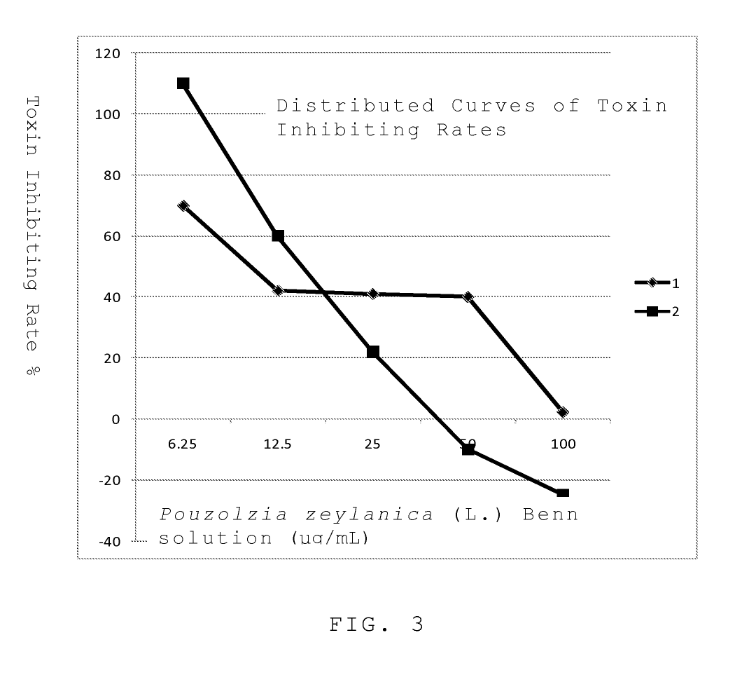

What is claimed is:

1. An ingredient extracted from Pouzolzia zeylanica (L.) Benn (0.00002%˜1% (w/w)) for anti-acne effect and manufactured with the following steps:
(1) Development of a Pouzolzia zeylanica (L.) Benn extract: Leaves of Pouzolzia zeylanica (L.) Benn with deionized or distilled water added are ground to develop a Pouzolzia zeylanica (L.) Benn solution which is further filtered to get a Pouzolzia zeylanica (L.) Benn extract;
(2) Manufacture of an anti-acne formula: The Pouzolzia zeylanica (L.) Benn extract is deposited in a low-temperature and dry environment (temperature between −20° C. and −48° C.; pressure between 10 K Bar and 133 K Bar) for development of a powdered anti-acne formula which is favorable to storage and portability;
(3) Development of an anti-acne solution: The powdered anti-acne formula with deionized or distilled water added is fully agitated and mixed to develop an anti-acne solution which is directly applied to acnes on skin.

2. An anti-acne formula manufactured with the ingredient extracted from Pouzolzia zeylanica (L.) Benn according to claim 1 wherein the anti-acne formula with citronella (0.5%˜1.5% (w/w)), compound disinfectant (0.3%˜1% (w/w)), and surfactant (0.5%˜3% (w/w)) added is developed to a fragrant bactericidal ointment featuring long-term storage.
